(19) United States
(12) Patent Application Publication
(10) Pub. No.: US 2015/0297083 A1
(43) Pub. Date: Oct. 22, 2015

(54) ELASTOGRAPHY DEVICE AND METHOD FOR OPERATING IT

(71) Applicants: Iva Cermakova, (US); Charité-Universitaetsmedizin Berlin, Berlin (DE)

(72) Inventors: Juergen BRAUN, Berlin (DE); Ingolf SACK, Berlin (DE); Sebastian HIRSCH, Berlin (DE); Ivo CERMAK, Chemnitz (DE)

(73) Assignees: CHARITÉ-UNIVERSITAETSMEDIZIN BERLIN, Berlin (DE); Iva Cermakova, Chemnitz (DE)

(21) Appl. No.: 14/688,589

(22) Filed: Apr. 16, 2015

(30) Foreign Application Priority Data
Apr. 17, 2014 (DE) 10 2014 207 513.0

(51) Int. Cl. A61B 5/00; A61B 5/055

(52) U.S. Cl. CPC A61B 5/0051; A61B 5/055

(57) ABSTRACT

The invention relates, inter alia, to a method for operating an elastography device (10), wherein an excitation unit (20) generates mechanical vibration patterns for generating mechanical tissue movements in human or animal tissue, and an image recording device (30) generates images of the tissue during the tissue movements.

According to the invention, it is provided that for initiating the respective next mechanical vibration pattern, a respective trigger signal (T) is fed into a trigger signal terminal (20a) of the excitation unit (20), and for configuring the excitation unit (20), configuration signals (K) are fed into the same trigger signal terminal (20a) of the excitation unit (20).

The invention relates to a method for operating an elastography device, wherein an excitation unit generates mechanical vibration patterns for generating mechanical tissue movements in human or animal tissue, and an image recording device generates images of the tissue during the tissue movements.

As is known, modern medical image recording devices, also called imaging modalities hereinafter,—such as, for example, magnetic resonance imaging (MRI) or sonography—allow, besides the representation of morphological soft-tissue contrast, unique possibilities for determining movement, flow and function in a living organism. One important application of MRI and sonography is the spatially resolved representation of viscoelastic characteristic variables of soft tissues, elastography, which allows sensitive image-aided palpation for identifying diseases. One major area of application for elastography by means of MRI and ultrasound is non-evasive graduation of liver fibrose [1, 2]. One main technical feature of elastography is the external mechanical stimulation of the tissue to be examined by means of an excitation unit. An excitation unit usually comprises a signal generator (or signal waveform generator), an amplifier and a suitable movement converter that performs the mechanical deformation of the tissue to be examined with the desired movement trajectory [3]. The external stimulation should be carried out as far as possible synchronously with the image recording in order to obtain information about the underlying mechanical properties of the tissue by means of the temporal control of the deformation patterns. The need for synchronization of the excitation unit with the image recording device gives rise to the problem of communication between these apparatuses with minimal latency or delay.

The invention is based on the object of specifying a method for operating an elastography device wherein communication between the image recording device and the excitation unit with as little delay as possible is achieved, in particular with regard to a possible change in the operating parameters during a measurement.

This object is achieved according to the invention by means of a method comprising the features as claimed in patent claim 1. Advantageous embodiments of the method according to the invention are specified in dependent claims.

According to the invention, it is accordingly provided that for initiating the respective next mechanical vibration pattern, a respective trigger signal is fed into a trigger signal terminal of the excitation unit, and for configuring the excitation unit, configuration signals are fed into the same trigger signal terminal of the excitation unit.

A major advantage of the method according to the invention is that one and the same signal line is used for the triggering and configuration of the excitation unit. Since signal lines intended for transmitting trigger signals, that is to say trigger signal lines, have to ensure per se a low-latency (low-delay) or latency-free (delay-free) signal transmission, just by virtue of the double utilization of the trigger signal line according to the invention a low-latency or latency-free signal transmission is also ensured for the configuration signals, and moreover in an advantageous manner without additional hardware costs.

It is regarded as particularly advantageous if the excitation unit evaluates signals present at the trigger signal terminal by means of an evaluation device and distinguishes trigger signals for initiating the respective next vibration pattern from configuration signals. In the case where a configuration signal is input, the configuration defined in the configuration signal is performed by the excitation unit.

The evaluation device preferably forwards trigger signals present at the trigger signal terminal of the excitation unit for initiating the respective next vibration pattern to the trigger input of a signal generator of the excitation unit. The evaluation device forwards configuration signals present at the trigger signal terminal of the excitation unit, said configuration signals each selecting or defining a signal pattern stored in the excitation unit, in particular in the signal generator, from a group of signal patterns stored in the excitation unit, in particular in the signal generator, preferably to a selection input of the signal generator for the selection of the respective signal pattern.

The signal patterns are preferably harmonic signal patterns, that is to say sinusoidal signal patterns having an individually assigned (single) oscillation frequency, an individually assigned amplitude and an individually assigned temporal signal pattern length. Alternatively, the signal patterns can also be formed by a superposition of two or more harmonic or sinusoidal oscillation frequencies.

It can also be provided that the evaluation device forwards configuration signals present at the trigger signal terminal of the excitation unit, which configuration signals are each intended to bring about a temporal synchronization of an internal time base (clock) of the image recording device with an internal time base (clock) of the excitation unit, to the internal time base of the excitation unit. Alternatively, a separate synchronization line between the image recording device and the excitation unit can be used for a synchronization of the two internal time bases.

A trigger signal is distinguished from a configuration signal preferably on the basis of the pulse length and/or the pulse amplitude of the signal or of at least one first pulse of the respective signal.

The evaluation device preferably outputs status signals indicating the respective apparatus status of the excitation unit at the trigger signal terminal of the excitation unit and transmits them via the trigger signal terminal to the signal output of the image recording device. In this embodiment, therefore, a bidirectional signal transmission takes place between the trigger signal terminal of the excitation unit and the signal output of the image recording device; in order to avoid a data collision, the excitation unit preferably operates as a slave and the image recording device preferably operates as a master. By way of example, the image recording device can request data from the excitation unit by means of a pulse or a pulse sequence and wait for the data, without itself sending anything in that time.

The invention additionally relates to an elastography device equipped with an excitation unit for generating mechanical vibration patterns and for generating mechanical tissue movements in human or animal tissue, and an image recording device. According to the invention, it is provided that the image recording device has a signal output, which it can optionally operate as a trigger signal or configuration signal output and via which it can optionally feed trigger signals for initiating a mechanical vibration pattern or configuration signals into a trigger signal terminal of the excitation unit. The excitation unit has an evaluation device connected to the trigger signal terminal, said evaluation device being configured in such a way that it evaluates signals present at the trigger signal terminal and distinguishes trigger signals for initiating a mechanical vibration pattern from the configuration signals.

With regard to the advantages of the elastography device according to the invention, reference should be made to the above explanations in association with the method according to the invention, since the advantages of the method correspondingly apply to the elastography device according to the invention.

The excitation unit preferably has a signal generator and an electromechanical transducer connected to the signal generator, said electromechanical transducer converting electrical signal patterns of the signal generator into corresponding mechanical vibration pattern. A trigger input of the signal generator is preferably connected to a trigger signal output of the evaluation device.

The evaluation device is preferably embodied in such a way that it outputs trigger signals present at the trigger signal terminal of the excitation unit for initiating a mechanical vibration pattern at the trigger signal output of the evaluation device and feeds them via said output into the trigger input of the signal generator.

Moreover, the evaluation device is preferably embodied in such a way that it forwards configuration signals present at the trigger signal terminal of the excitation unit, said configuration signals each defining a signal pattern stored in the signal generator, via a configuration output to a selection input of the signal generator for the selection of the respective signal pattern.

It can also be provided that the evaluation device forwards configuration signals present at the trigger signal terminal, which configuration signals are each intended to bring about a temporal synchronization of an internal time base (clock) of the image recording device with an internal time base (clock) of the excitation unit, to the internal time base of the excitation unit. Alternatively, an additional synchronization line can also be present for synchronizing the time bases.

It is regarded as particularly advantageous if the evaluation device is embodied in such a way that, in the case of a signal present at the trigger signal terminal, it evaluates
the pulse length and/or the pulse amplitude of the respective signal or
the pulse length and/or the pulse amplitude of a first pulse (individual pulse) of the respective signal
   and establishes whether a trigger signal for initiating a mechanical vibration pattern or a configuration signal is involved, on the basis of the pulse length and/or the pulse amplitude.

The invention additionally relates to an excitation unit for an elastography device, in particular one such as has been described above. According to the invention, with regard to such an excitation unit, it is provided that the latter has an evaluation device connected to a trigger signal terminal of the excitation unit, said evaluation device being configured in such a way that it evaluates signals present at the trigger signal terminal and separates trigger signals for initiating a mechanical vibration pattern from configuration signals for configuring the excitation unit.

With regard to the advantages of the excitation unit according to the invention, reference should be made to the above explanations in association with the elastography device according to the invention, since the advantages of the elastography device according to the invention correspond to those of the excitation unit according to the invention.

The invention additionally relates to an image recording device for an elastography device, in particular one such as has been described above. According to the invention, with regard to such an image recording device, it is provided that the latter has a signal output, which it can optionally operate as a trigger signal or configuration signal output and via which it can optionally feed trigger signals for initiating a mechanical vibration pattern or configuration signals for the configuration of an excitation unit into a trigger signal terminal of the excitation unit.

With regard to the advantages of the image recording device according to the invention, reference should be made to the above explanations in association with the elastography device according to the invention, since the advantages of the elastography device according to the invention correspond to those of the image recording device according to the invention.

The invention is explained in greater detail on the basis of exemplary embodiments; in the figures here, by way of example:

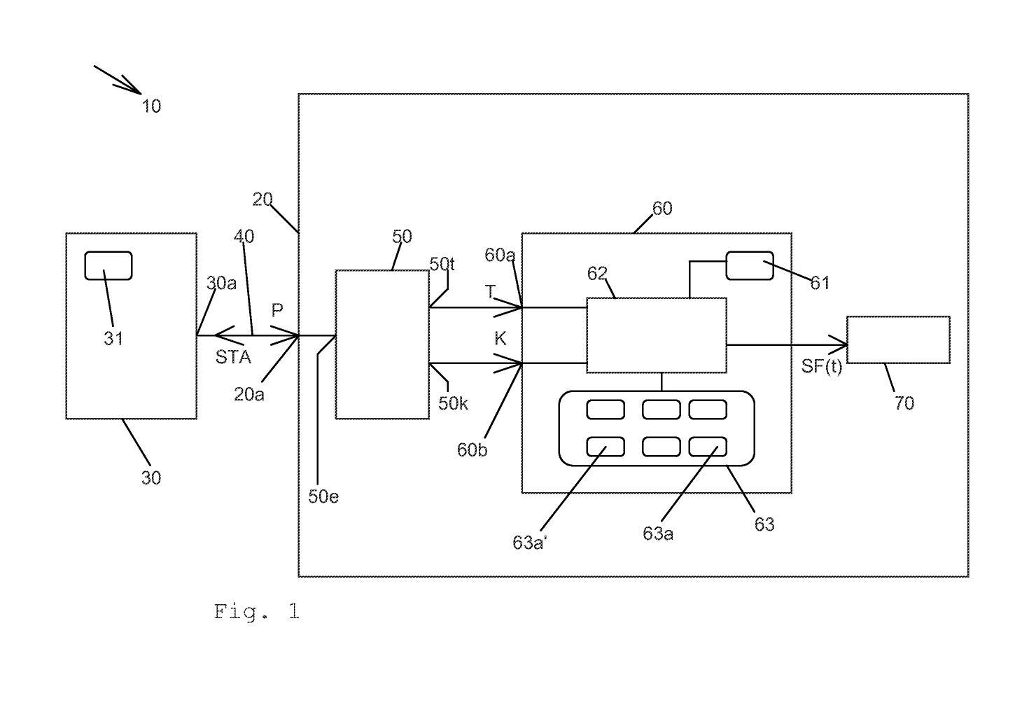

FIG. 1 shows a first exemplary embodiment of an elastography device according to the invention, on the basis of which by way of example one embodiment variant of the method according to the invention is also elucidated,

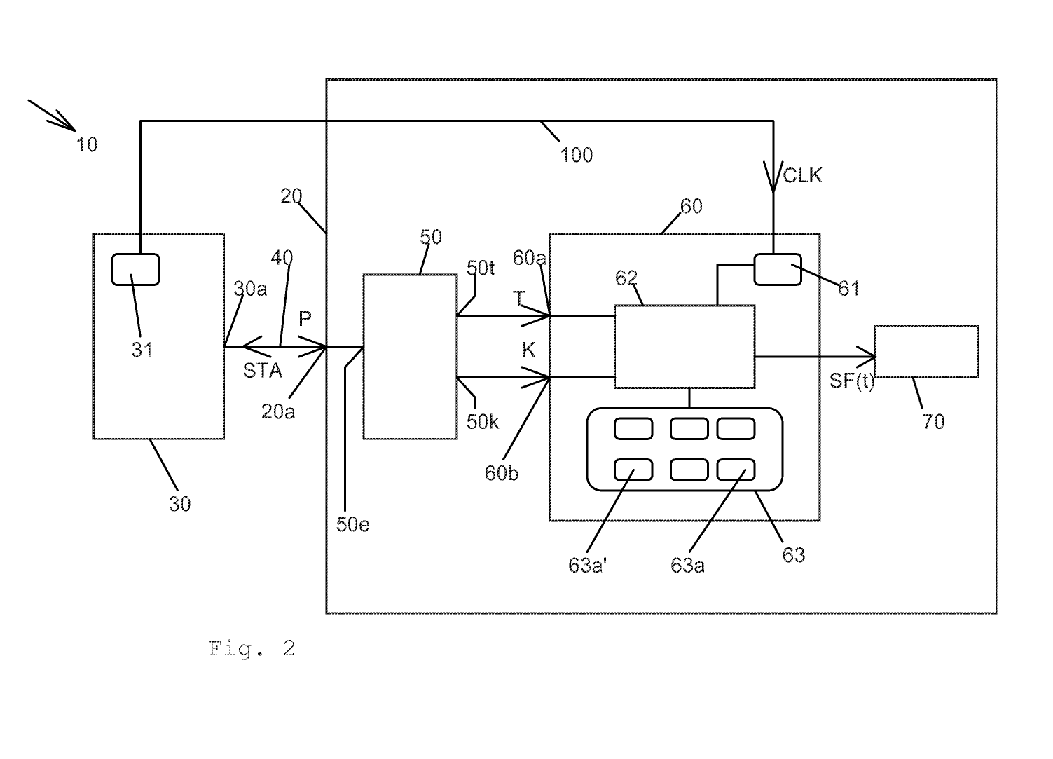

FIG. 2 shows a second exemplary embodiment of an elastography device according to the invention, on the basis of which by way of example another embodiment variant of the method according to the invention is elucidated, and

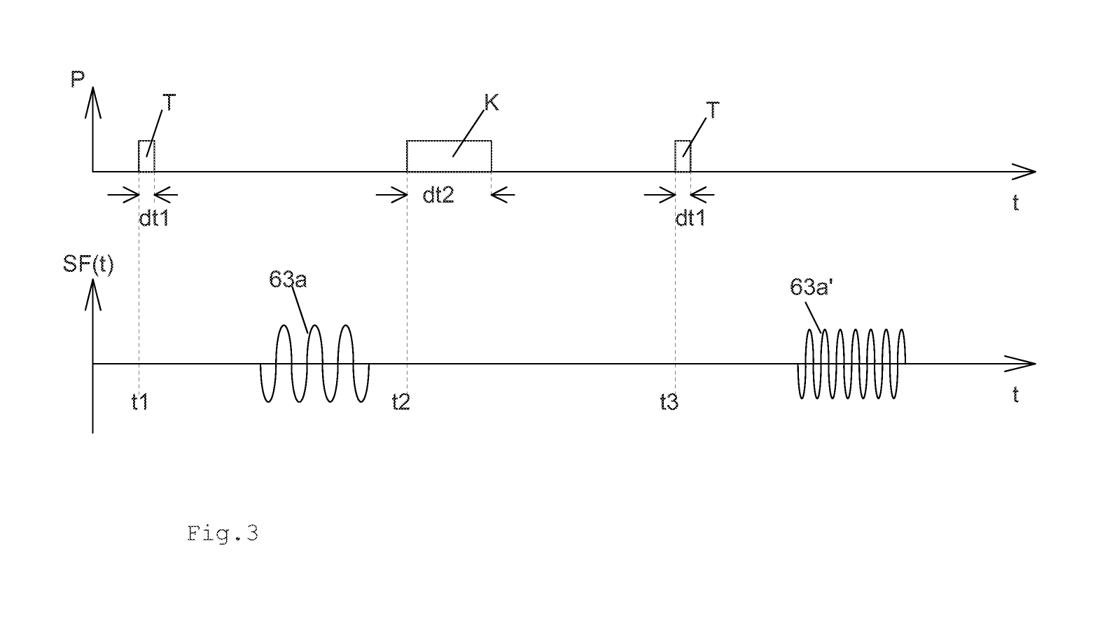

FIG. 3 shows by way of example the time profile of control pulses P at the trigger signal terminal of an evaluation unit of the elastography device in accordance with FIGS. 1 and 2 and also the temporal profile of the electrical signal patterns output by a signal generator of the elastography device for driving an electromechanical transducer.

FIG. 1 shows one exemplary embodiment of an elastography device 10. The elastography device 10 comprises an excitation unit 20 for generating mechanical vibration patterns and for generating mechanical tissue movements in human or animal tissue. Furthermore, the elastography device 10 comprises an image recording device 30.

A signal output 30a of the image recording device 30 is connected to a trigger signal terminal 20a of the excitation unit 20 via a signal line 40. Trigger signals T for triggering the excitation unit 20 and for generating the respective next mechanical vibration pattern can be fed in via said trigger signal terminal 20a and thus via the signal line 40. Furthermore, configuration signals K, for the configuration of the excitation unit 20 can be transmitted via the trigger signal terminal 20a and the signal line 40.

The signal line 40 can be an electrical or optical line; alternatively, instead of the signal line 40, a wireless connection (e.g. radio connection) can be used as communication connection or communication channel.

The excitation unit 20 has an evaluation device 50 connected to the trigger signal terminal 20a, said evaluation device being embodied in such a way that it evaluates signals present at the trigger signal terminal 20a and distinguishes trigger signals T for initiating mechanical vibration pattern from configuration signals K.

The excitation unit 20 additionally comprises a signal generator 60 and an electromechanical transducer 70 connected to the signal generator 60, said electromechanical transducer converting electrical signal patterns of the signal generator 60 into corresponding mechanical vibration patterns.

A trigger input 60a of the signal generator 60 is connected to a trigger signal output 50t of the evaluation device 50, such that the evaluation device 50 can forward trigger signals T present at the trigger signal terminal 20a of the excitation unit 20 for initiating the respective next mechanical vibration pattern via the trigger signal output 50t to the trigger input 60a of the signal generator 60.

The evaluation device 50 separates trigger signals T present at the trigger signal terminal 20a of the excitation unit 20 from configuration signals K present there, said configuration signals selecting or defining for example a signal pattern stored in the signal generator 60, and forwards them via a configuration output 50k to a selection input 60b of the signal generator 60.

In the case of the exemplary embodiment in accordance with FIG. 1, the evaluation device 50 can additionally forward configuration signals K, which are each intended to bring about a temporal synchronization of an internal clock 31 of the image recording device 30 with an internal clock 61 of the excitation unit 20, to the internal clock 61 via a control device 62 of the signal generator 60. The internal clock 61 can be arranged in the signal generator 60, as illustrated in FIG. 1, or can be a component which is separated therefrom and which interacts with the signal generator 60 and predefines the operating clock signal thereof.

In the case of the exemplary embodiment in accordance with FIG. 1, the signal generator 60 comprises, besides the clock 61 and the control device 62, which can be a computing device for example in the form of a microprocessor, a memory 63, in which a multiplicity of signal patterns 63a are stored. One of said signal patterns 63a is selected by the evaluation device 50 by the inputting of a corresponding configuration signal K at the selection input 60b of the signal generator 60.

The evaluation device 50 is preferably embodied in such a way that, in the case of a signal present at the trigger signal terminal 20a of the excitation unit 20 or at the trigger signal input 50e of the evaluation device 50, it evaluates the pulse length and/or the pulse amplitude of a first pulse of the respective signal and establishes whether a trigger signal T for initiating a vibration pattern or a configuration signal K is involved, on the basis of the pulse length and/or the pulse amplitude of the first pulse of the respective signal.

The functioning of the elastography device 10 in accordance with FIG. 1 will be explained in greater detail by way of example for the case where time-harmonic elastography by means of MRI as image recording device 30 is intended to be carried out:

The repetition time of the image recording is intended to be 100 ms, for example, i.e. the vibration signal generated by the signal generator 60 must be repeated exactly every 100 ms in order to avoid interference between image recording and object movement (tissue material). Furthermore, the phase of the waveform is intended to be shifted within the 100 ms repetition time, in order to achieve a controlled passage of the wave in the MRI image [3]. In addition, after the recording of these phase steps of the shear wave at an individual harmonic excitation frequency, a further excitation frequency with an appropriate number of periods and amplitude is intended to be selected in the signal generator in order to carry out the subsequent elastography examination with an altered time-harmonic stimulus. It is only the combined evaluation of all the time-harmonic wave fields that makes it possible to calculate particularly high-resolution maps of the viscoelastic properties in the tissue. By contrast, even small deviations between the periodic behavior of vibration signal and image recording can lead to an erroneous sampling of the tissue oscillation and thus render the elastography examination wholly or partly unusable.

With regard to this problem, in the case of the exemplary embodiment in accordance with FIG. 1, a low-delay, that is to say instantaneous or quasi-instantaneous, information transmission between the image recording device 30 and the excitation unit 20 is made possible which is able

(i) to select the vibration signal with desired frequency, number of periods and amplitude by means of the configuration signal K,
(ii) to initiate it, i.e. to process it in the signal generator 60, to insert a controlled temporal offset in the process, in order to bring about different phases of the vibration in the tissue at the time of the image recording, and
(iii) to perform the resynchronization of the vibration signal with the image recording in the further course of the examination.

In the method described in greater detail by way of example below, the communication between the image recording device 30 and the excitation unit 20 takes place via the signal line 40, namely by means of threshold-value-based signals of different length and/or number for transferring coded information. Since these signals code additional information, they are designated hereinafter as “control pulses” P. The control pulses P can have different durations, which the excitation unit 20 converts into information for the selection of a series of possible actions. Possible actions of the excitation unit 20 which can be initiated by the control pulses P consist in selection, configuration, initiation, synchronization and termination of individual vibration signal waveforms and arbitrary combinations of these actions.

In addition, for the purpose of an advantageous extension of the method, a reverse communication from the excitation unit 20 to the image recording device 30 via the signal line 40 can take place, for example by means of status signals such as are identified by the reference sign STA in FIG. 1. This communication path can be utilized for example for indicating the readiness of the excitation unit 20 or the safety-governed termination of the image recording. Furthermore, the excitation unit 20 can thereby confirm the proper processing of a command sent previously and thus reduce the risk of an incorrect configuration.

The exemplary embodiment shown in FIG. 1 for the communication for configuration and synchronization between an MRI apparatus and an excitation unit 20 by means of control pulses can be used for example in multifrequency MR elastography (MMRE). By way of example, up to one hundred signal patterns 63a in the form of protocols (preferably consisting of a vibration signal waveform, its duration, amplitude and the number of cycles to be output) can be stored beforehand in the memory 63. Each protocol is preferably identified by means of a numeral index n in the range of between 0 and 99.

The signal patterns 63a are preferably harmonic signal patterns, that is to say sinusoidal signal patterns having an individually assigned (single) oscillation frequency, an individually assigned amplitude and an individually assigned temporal signal pattern length. In other words, the signal patterns 63a stored in the memory 63 preferably differ only with regard to their (single) oscillation frequency, their amplitude and their signal pattern length.

During an examination, the duration of each control pulse P is evaluated by the evaluation device 50: short pulses having a duration of 10 microseconds are interpreted for example as “regular” trigger pulses T for triggering and accordingly lead to outputting of the currently configured waveform.

Pulses having a duration exceeding 15 microseconds, for example, serve for example for communication for the configuration of the signal generator 60. Their duration dt is translated into a protocol index n by the evaluation device 50 for example according to the following formula:

n=floor((dt−15 μs)/10 μs). (1)

In such a case, the signal generator 60 reads out the setting stored in the protocol “n”, that is to say the respectively corresponding signal pattern 63a from the memory 63, and configures the active signal waveform accordingly.

In the case of the advantageous exemplary embodiment mentioned above, therefore, a control pulse P identified as configuration signal K does not lead directly to the outputting of a waveform; the respective next vibration is initiated only after successful configuration and after arrival of the next control pulse P identified as trigger signal T.

On the part of the image recording device 30, that is to say here the MRI apparatus, the measurement parameters are preferably additionally adapted to the respective current configuration of the signal generator 60 or the signal pattern 63a respectively selected. By way of example, it is possible for the gradient form used in the MR sequence for movement coding to be adapted to the frequency of the vibration generated by the excitation unit 20, in order to obtain maximum movement sensitivity. An adjustment of the configuration of the image recording device 30 and of the configuration table in the signal generator 60 can thus ensure that each measurement is carried out with parameters optimally coordinated with one another. As a result, firstly, the probability of an incorrect measurement as a result of an operating error is reduced; secondly, the progression of an examination is also accelerated because manual changeover of the measurement parameters between the individual measurements is omitted.

In order to achieve an optimum insensitivity toward fluctuations of the length of the control pulses P, the algorithms for the coding and decoding of the index n preferably differ. While the decoding is carried out by the signal generator 60 for example according to equation (1) mentioned above, the pulse duration is converted in the image recording device 30 for example in accordance with

dt=20 μs+n·10 μs (2)

The different offsets of 15 and 20 microseconds in equation (1) and (2) should be mentioned here. A tolerance of 5 microseconds in both directions (i.e. in the direction of excessively long and excessively short control pulses) is achieved as a result. For example, the setting n=1 would be coded as 30 μs by the image recording device 30 in accordance with equation (2), while according to equation (1) every pulse duration in the range of 25 μs≦dt<35 μs would be decoded as n=1.

FIG. 3 shows by way of example the time profile of control pulses P against time t, which control pulses can arrive at the trigger signal terminal 20a of the excitation unit 20 and thus at the trigger signal input 50e of the evaluation device 50, and also the time profile of signal patterns SF(t) output to the electromechanical transducer 70 by the signal generator 60.

FIG. 3 shows, for example, how at the instant t1 a trigger signal T arrives at the trigger signal terminal 20a of the excitation unit. The evaluation device 50 identifies the trigger signal T in the control pulse P on the basis of the temporal length dt1 of the pulse and forwards the trigger signal T to the trigger input 60a of the signal generator 60. By way of example, the evaluation device 50 will identify the trigger signal T in the control pulse P if the temporal length dt1 of the pulse is a maximum of 15 μs.

Upon receiving the trigger signal T, the signal generator 60 generates an electrical signal pattern 63a on the output side. The signal pattern 63a was set in a previous configuration step in the signal generator 60 or read out from the memory 63 and activated by corresponding setting of the harmonic oscillation frequency, the amplitude and the oscillation duration.

A control pulse P that forms a configuration signal K arrives at the trigger signal terminal 20a of the excitation unit 20 at the instant t2 in the case of the example in accordance with FIG. 3. The evaluation device 50 identifies the configuration signal K in the control pulse P on the basis of the temporal length dt2 of the pulse and forwards the configuration signal K to the selection input 60b of the signal generator 60. For example, the evaluation device 50 will identify the configuration signal K in the control pulse P if the temporal pulse length dt2 is a minimum of 15 μs.

Upon receiving the configuration signal K, the signal generator 60 changes over the signal pattern to the new signal pattern 63a′ by reading out the new signal pattern 63a′ from the memory 63 and activating it and by correspondingly resetting the harmonic oscillation frequency, the amplitude and the oscillation duration.

A trigger signal T arrives again at the trigger signal terminal 20a of the excitation unit 20 at the instant t3 in the case of the example in accordance with FIG. 3. The evaluation device 50 identifies the trigger signal T in the control pulse P on the basis of the temporal length dt1 of the pulse and accordingly forwards the trigger signal T to the trigger input 60a of the signal generator 60. For example, the evaluation device 50 will identify the trigger signal T in the control pulse P if the temporal length dt1 is a maximum of 15 μs.

Upon receiving the trigger signal T, the signal generator 60 generates on the output side the signal pattern 63a′ newly set beforehand.

In the case of the exemplary embodiment in accordance with FIG. 3, the control pulses P are positive pulses; alternatively, negative or inverted pulses can also be used.

The delay—shown in the case of the exemplary embodiment in accordance with FIG. 3—between the inputting of the control pulses P, in particular the inputting of the trigger signals T, and the outputting of the signal patterns 63a or 63a′ should be understood to be merely by way of example; the delay can be variable and, apart from internal propagation delays in the evaluation device 50 and the signal generator 60, can also be zero, for example.

Advantages of the above-described elastography device 10 and the operating method thereof consist in
a time-saving, automated real-time control and configuration of the signal generator 60 of the elastography device 10,
the provision of a unidirectional or bidirectional communication channel via a single signal line 40 with minimal latency for transmitting trigger signals T and configuration signals K by means of control pulses P,
a cost-effective implementability by the use of established standards and interfaces (for example: TTL pulses via coaxial cable, or optical pulses via optical waveguide),
a simple subsequent extendibility of the supported command set by modification of the action table stored at the receiver end and software adaptation in the image recording device 30 (MRI apparatus), and
an extendibility to bidirectional communication by addition of a second, independent unidirectional communication channel.

FIG. 2 shows a further exemplary embodiment of an elastography device 10. The elastography device 10 substantially corresponds to the elastography device 10 in accordance with FIG. 1 and differs therefrom only with regard to the temporal synchronization of the internal clock 31 of the image recording device 30 with the internal clock 61 of the excitation unit 20. A separate synchronization line 100 is provided for the synchronization of the two clocks 31 and 61, and enables a direct exchange of synchronization signals CLK.

For the rest, the above explanations concerning FIG. 1 correspondingly apply to the exemplary embodiment in accordance with FIG. 2.

In the case of the exemplary embodiments in accordance with FIGS. 1 to 3, the distinguishing of trigger signals and configuration signals and also the identifying of signal patterns 63a in configuration signals are carried out solely on the basis of the pulse length of the control pulses P; alternatively, a coding of the signal patterns 63a can also be carried out in some other way, for example by means of the amplitude of the control pulses or by means of binary coded control pulse sequences in which, for example, in each case the first pulse (start pulse) and/or further pulses of the control pulse sequence enable(s) the distinguishing between trigger signal and configuration signal and the further pulses of the respective control pulse sequences determine the desired configuration.

### LITERATURE

[1] Venkatesh S K, Yin M, Ehman R L. Magnetic resonance elastography of liver: Technique, analysis, and clinical applications. J. Magn Reson Imaging 2013; 37(3): 544-555.
[2] Bamber J, Cosgrove D, Dietrich C F, Fromageau J, Bojunga J, Calliada F, Cantisani V, Correas J M, D'Onofrio M, Drakonaki E E, Fink M, Freidrich-Rust M, Gilja O H, Havre R F, Jenssen C, Klauser A S, Ohlinger R, Saftoiu A, Schaefer F, Sporea I, Piscaglia F. EFSUMB guidelines and recommendations on the clinical use of ultrasound elastography. Part 1: Basic principles and technology. Ultraschall Med 2013; 34(2): 169-184.
[3] Hirsch S, Guo J, Reiter R, Papazoglou S, Kroencke T, Braun J, Sack I. MR Elastography of the Liver and the Spleen Using a Piezoelectric Driver, Single-Shot Wave-Field Acquisition, and Multifrequency Dual Parameter Reconstruction. Magn Reson Med 2013; 71(1): 267-277.

### REFERENCE SIGNS

10 Elastography device
20 Excitation unit
20a Trigger signal terminal of the excitation unit
30 Image recording device
30a Signal output of the image recording device
31 Internal clock
40 Signal line
50 Evaluation device
50e Trigger signal input
50k Configuration output
50t Trigger signal output
60 Signal generator
60a Trigger input of the signal generator
60b Selection input of the signal generator
61 Internal clock
62 Control device
63 Memory
63a Signal pattern
63a′ Signal pattern
70 Electromechanical transducer
100 Synchronization line
dt Duration
dt1 Pulse length
dt2 Pulse length
CLK Synchronization signals
K Configuration signals
P Control pulses
SF(t) Time profile of signal patterns
STA Status signals
t Time
t1 Instant
t2 Instant
t3 Instant
T Trigger signals/trigger pulses

1. A method for operating an elastography device (10), wherein
an excitation unit (20) generates mechanical vibration patterns for generating mechanical tissue movements in human or animal tissue, and
an image recording device (30) generates images of the tissue during the tissue movements,
wherein
for initiating the respective next mechanical vibration pattern, a respective trigger signal (T) is fed into a trigger signal terminal (20a) of the excitation unit (20), and
for configuring the excitation unit (20), configuration signals (K) are fed into the same trigger signal terminal (20a) of the excitation unit (20).

2. The method as claimed in claim 1,
wherein
the excitation unit (20) evaluates signals present at the trigger signal terminal (20a) by means of an evaluation device (50) and distinguishes trigger signals (T) for initiating the respective next mechanical vibration pattern from configuration signals (K), and
in the case where a configuration signal (K) is input, the configuration defined in the configuration signal (K) is performed by the excitation unit (20).

3. The method as claimed in claim 2,
wherein
the evaluation device (50) forwards trigger signals (T) present at the trigger signal terminal (20a) for initiating the respective next mechanical vibration pattern to a trigger input (60a) of a signal generator (60) of the excitation unit (20), and
the evaluation device (50) forwards configuration signals (K) present at the trigger signal terminal (20a), said configuration signals each defining a signal pattern (63a) stored in the signal generator (60) from a group of signal patterns (63a) stored in the signal generator (60), to a selection input (60b) of the signal generator (60) for the selection of the respective signal pattern (63a).

4. The method as claimed in claim 2,
wherein
the evaluation device (50) forwards configuration signals (K) present at the trigger signal terminal (20a), which configuration signals are each intended to bring about a temporal synchronization of an internal time base of the image recording device (30) with an internal time base (31) of the excitation unit (20), to the internal time base (61) of the excitation unit (20).

5. The method as claimed in claim 1,
wherein
a trigger signal (T) is distinguished from a configuration signal (K) on the basis of the pulse length and/or the pulse amplitude and/or the pulse pattern of the respective signal or of at least one first pulse of the respective signal.

6. The method as claimed in claim 1,
wherein
the evaluation device (50), for transmitting the apparatus status of the excitation unit (20), outputs status signals (STA) at the trigger signal terminal (20a) and transmits them via said trigger signal terminal (20a) to the image recording device (30).

7. An elastography device (10) comprising
an excitation unit (20) for generating mechanical vibration patterns and for generating mechanical tissue movements in human or animal tissue, and
an image recording device (30),
wherein
the image recording device (30) has a signal output (30a), which it can optionally operate as a trigger signal or configuration signal output and via which it can optionally feed trigger signals (T) for initiating a mechanical vibration pattern or configuration signals (K) into a trigger signal terminal (20a) of an excitation unit (20), and
the excitation unit (20) has an evaluation device (50) connected to the trigger signal terminal (20a), said evaluation device being configured in such a way that it evaluates signals present at the trigger signal terminal (20a) and distinguishes trigger signals (T) for initiating a mechanical vibration pattern from configuration signals (K).

8. The elastography device (10) as claimed in claim 7,
wherein
the excitation unit (20) has a signal generator (60) and an electromechanical transducer (70) connected to the signal generator (60), said electromechanical transducer converting electrical signal patterns of the signal generator (60) into corresponding mechanical vibration patterns,
a trigger input (60a) of the signal generator (60) is connected to a trigger signal output (50t) of the evaluation device (50) and the evaluation device (50) forwards trigger signals (T) present at the trigger signal terminal (20a) for initiating a mechanical vibration pattern to the trigger input (60a) of the signal generator (60), and
a selection input (60b) of the signal generator (60) is connected to a configuration output (50k) of the evaluation device (50) and the evaluation device (50) forwards configuration signals (K) present at the trigger signal terminal (20a), said configuration signals each selecting a selected signal pattern (63a) stored in the signal generator (60), via the configuration output (50k), to the selection input (60b) of the signal generator (60) for the selection of the respective signal pattern (63a),
wherein the evaluation device (50) is embodied in such a way that, in the case of a signal present at the trigger signal terminal (20a), it evaluates the pulse length and/or the pulse amplitude of the respective signal or the pulse length and/or the pulse amplitude of a first pulse of the respective signal and establishes whether a trigger signal (T) for initiating a mechanical vibration pattern or a configuration signal (K) is involved, on the basis of the pulse length and/or the pulse amplitude.

9. An excitation unit (20) for an elastography device (10), in particular one such as claimed in claim 7, wherein
the excitation unit (20) has an evaluation device (50) connected to a trigger signal terminal (20a) of the excitation unit (20), said evaluation device being configured in such a way that it evaluates signals present at the trigger signal terminal (20a) and separates trigger signals (T) for initiating a mechanical vibration pattern from configuration signals for configuring the excitation unit (20).

10. An image recording device (30) for an elastography device (10), in particular one such as claimed in claim 7,
wherein
the image recording device (30) has a signal output (30a), which it can optionally operate as a trigger signal or configuration signal output and via which it can optionally feed trigger signals (T) for initiating a mechanical vibration pattern or configuration signals (K) into a trigger signal terminal (20a) of an excitation unit (20).
